(19) United States
(12) Patent Application Publication
(10) Pub. No.: US 2025/0361316 A1
(43) Pub. Date: Nov. 27, 2025

(54) PEPTIDE, ANTIBODY OR ANTIGEN-BINDING FRAGMENT THEREOF SPECIFICALLY BINDING TO ACE2 RECEPTOR, AND COMPOSITIONS FOR PREVENTING SARS-COV-2 CONTAINING THE SAME

(71) Applicant: UIF (University Industry Foundation), Yonsei University, Seoul (KR)

(72) Inventor: Jae-Chul PYUN, Seoul (KR)

(73) Assignee: UIF (University Industry Foundation), Yonsei University, Seoul (KR)

(21) Appl. No.: 19/213,395

(22) Filed: May 20, 2025

(30) Foreign Application Priority Data
May 22, 2024 (KR) 10-2024-0066415

(51) Int. Cl. C07K 16/28

(52) U.S. Cl. CPC C07K 16/2896; C07K 2317/14; C07K 2317/565; C07K 2317/76; C07K 2317/92

(57) ABSTRACT

Disclosed are a peptide, an antibody, or an antigen-binding fragment thereof, which specifically binds to an ACE2 (angiotensin-converting enzyme 2) receptor, and a composition for preventing SARS-CoV-2, the composition comprising the same. The peptide includes at least one peptide sequence selected from a group consisting of [SEQ ID NO: 1] GHPVNSVLLDF, [SEQ ID NO: 2] GHPRVNVGGDF, [SEQ ID NO: 3] GVLGPRLLIDY and [SEQ ID NO: 4] DGPINRTTIDY.

### CROSS-REFERENCE TO RELATED APPLICATION(S)

This application claims the benefit under 35 USC 119 (a) of Korean Patent Application No. KR 10-2024-0066415 filed on May 22, 2024, with the Korean Intellectual Property Office, the disclosures of which are incorporated herein by reference in their entirety.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_1710950011_SequenceListing” and is created on May 20, 2025, contains a 6.33 KB file.

### BACKGROUND

### Field

The present disclosure relates to a peptide, an antibody, or an antigen-binding fragment thereof, which specifically binds to an ACE2 (angiotensin-converting enzyme 2) receptor, and a composition for preventing SARS-COV-2, the composition comprising the same.

### Description of Related Art

SARS-COV-2 is a genome-sized 30 kb RNA virus that encodes four structural proteins: replicase and spike protein (SP), envelope protein (SP), membrane protein (MP), and nucleocapsid protein (NP). SP has become a target for vaccine development as it is known to cause viral infection by binding to angiotensin-converting enzyme 2 (ACE2) receptor in the host cells. As another approach to prevent infection of SARS-COV-2, it has been attempted to block viral attachment to host cells using antibodies against SP of the ACE2 receptor of the host cell.

### SUMMARY

In accordance with the present disclosure, Fv-antibodies against the ACE2 receptor were screened in the Fv-antibody library to prevent SARS-COV-2 infection by blocking the attachment of the virus to the host cell (FIG. 1).

FIG. 1 is a diagram showing the screening of Fv-antibodies and the mechanism of blocking SARS-COV-2 infection. A first part of FIG. 1 shows the process in which Fv-antibodies with randomized CDR3 sites are screened and expressed in a Fv-antibody library with E. coli. A second part of FIG. 1 shows that the screened Fv-antibody binds to the ACE2 receptor, and in particular, this binding is expressed in a form bound to tdTomato. Referring to FIG. 1, how a technical approach for selecting an antibody having a specific amino acid sequence having a high affinity to the ACE2 receptor is performed in the antibody selection process. A process in which the specific antibody is expressed in E. coli using Fv-antibody libraries, and then binds to the ACE2 receptor is well described. Referring to FIG. 1, the advantages or effects of the subject matters of the present disclosure that may effectively block the spike protein of the SARS-COV-2 virus from binding to the receptor of the host cell by binding the selected Fv-antibody to the ACE2 receptor are identified.

The Fv-antibody contains a heavy chain variable (V_{H}) region of immunoglobulin G, which is composed of three complementarity-determining regions (CDRs) and four framework regions (FRs). The Fv-antibody library was prepared using site-directed mutagenesis of the CDR3 region (11 residues). Then, autodisplay technology was utilized to express the Fv-antibody library on the outer membrane of E. coli as shown in FIG. 2A. The surface density of Fv-antibodies in the prepared Fv-antibody library was greater than 10⁵ Fv-antibodies/clone, and the expression yield was more than 90% of E. coli within the total library population. The diversity of the library was estimated to be more than 10⁶ Fv-antibodies/library. Because of this high expression yield, the Fv-antibody library has been used for screening CDR3 sequences with high affinity for target analytes, such as monoamine oxidase-A (MAO-A), monoamine oxidase-B (MAO-B), and monocarboxylate transporter-1, without repeated panning processes.

A purpose to be achieved by the present disclosure is to prevent the spread of the virus by blocking the process in which the SARS-COV-2 virus binds to the ACE2 receptor of the host cell and causes infection. In this process, the selected peptide sequences specifically bind to the ACE2 receptor, thereby competitively interfering with the binding of the spike protein to the receptor.

One aspect of the present disclosure provides a peptide capable of specifically binding to an ACE2 (angiotensin-converting enzyme 2) receptor, the peptide comprising at least one peptide sequence selected from a group consisting of a peptide sequence of SEQ ID NO: 1, a peptide sequence of SEQ ID NO: 2, a peptide sequence of SEQ ID NO: 3, and a peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY. |

Another aspect of the present disclosure provides a nucleic acid coding the peptide as described above.

Still another aspect of the present disclosure provides a recombinant expression vector including the nucleic acid as described above.

Still yet another aspect of the present disclosure provides a cell transformed with the recombinant expression vector as described above.

In one embodiment, the cell includes at least one cell selected from the group consisting of animal cells, plant cells, yeast, E. coli, and insect cells.

In one embodiment, the cell includes at least one cell selected from the group consisting of COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

Still yet another aspect of the present disclosure provides an antibody or antigen-binding fragment thereof capable of specifically binding to an ACE2 receptor, wherein the antibody includes a Fv antibody, wherein a CDR3 region of the Fv antibody includes at least one peptide sequence selected from the group consisting of a peptide sequence of SEQ ID NO: 1, a peptide sequence of SEQ ID NO: 2, a peptide sequence of SEQ ID NO: 3, and a peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY. |

Still yet another aspect of the present disclosure provides a composition for preventing SARS-COV-2, the composition comprising the antibody or antigen-binding fragment thereof as described above.

Specifically, in accordance with the present disclosure, for the prevention of SARS-CoV-2 infection, four Fv-antibodies with binding affinity for the ACE2 receptor were screened from an Fv-antibody library. The screened Fv-antibodies were expressed as soluble proteins and estimated to have a high binding affinity, comparable to that between SARS-COV-2 and the ACE2 receptor. The interaction between the Fv-antibodies and the ACE2 receptor was analyzed using docking simulation, and the significant binding affinity of the screened Fv-antibodies was attributed to the homology in amino acid sequence with the ACE2 receptor. The neutralizing activities of the Fv-antibodies were demonstrated using a cell-based infection assay based on four pseudo-virus types with SARS-COV-2 variant spike proteins (Wildtype D614, Delta B.1.617.2, and Omicron BA.2, and Omicron BA.4/5).

The effect of the present disclosure is that the introduction of a peptide that specifically binds to the ACE2 receptor can significantly reduce the ability of the virus to penetrate host cells. These peptides can effectively block binding between viral spike proteins and the receptors, contributing to the prevention of viral spread in the early stages of infection. In addition, this approach has the possibility of flexibly responding to future virus mutations, and thus may be effective against mutant viruses.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

FIG. 1 is a diagram showing the screening of Fv-antibodies and the mechanism of blocking SARS-COV-2 infection.

FIG. 2A Screening Fv-antibodies from the Fv-antibody library with the binding affinity to the ACE2 receptor: Preparation of the Fv-antibody library through site-directed mutagenesis and autodisplay of the library on the outer membrane of E. coli.

FIG. 2B Screening Fv-antibodies from the Fv-antibody library with the binding affinity to the ACE2 receptor: Screening of target clones with binding affinity to the ACE2 probe using a screening gate.

FIG. 2C Screening Fv-antibodies from the Fv-antibody library with the binding affinity to the ACE2 receptor: Flow cytometric analysis of the ACE2 probe activity in screened clones.

FIG. 3A Binding analysis of expressed Fv-antibodies: Expression of screened Fv-antibodies.

FIG. 3B Binding analysis of expressed Fv-antibodies: Binding affinity of expressed Fv-antibodies to cells that overexpressed ACE2 receptor.

FIG. 3C Binding analysis of expressed Fv-antibodies: Binding sites of Fv-antibodies on ACE2 receptors.

FIG. 3D Binding analysis of expressed Fv-antibodies: Homology analysis between SARS-COV-2 RBD and anti-ACE2r Fv-antibodies.

FIG. 4A In vitro cell-based infection assay: (a) In vitro cell-based infection assay using cells that overexpress the ACE2 receptor and pseudo-virus particles with SP from SARS-COV-2 variants.

FIG. 4B In vitro cell-based infection assay: Neutralizing activity of screened Fv-antibodies. The concentration of Fv-antibodies was set to their binding affinity (KD), and the Fv antibodies were incubated for 48 h.

FIG. 4C In vitro cell-based infection assay: Estimation of the inhibition ratio of SARS-CoV-2 infection. Statistical significance is denoted as follows: *P<0.05, **P<0.01, and ***P <0.001.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In interpreting a numerical value, the value is interpreted as including an error range unless there is no separate explicit description thereof. In the context of the present disclosure, the term “about” may mean about ±1%, about ±2%, about ±3%, about ±4%, about ±5%, about ±6%, about ±7%, about ±8%, about ±9%, or about ±10% of a value stated herein.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A peptide according to an embodiment of the present disclosure is a peptide capable of specifically binding to a ACE2 receptor, and may include one or more peptide sequences selected from the group consisting of a peptide sequence of SEQ ID NO: 1, a peptide sequence of SEQ ID NO: 2, a peptide sequence of SEQ ID NO: 3, and a peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY |

In the context of the present disclosure, the meaning of the ACE2 receptor is an acronym of an angiotensin-converting enzyme 2, which is a protein present on a host cell surface. The spike protein of SARS-COV-2 virus binds to this receptor. Thus, this receptor plays an important role in the process of the virus invasion into cells. That is, this receptor enables the infection by providing a pathway for entry of the virus into the cells. The ACE2 is widely distributed on the cell surface of several organs, mainly the heart, kidneys, and lungs. Via the binding of the SARS-COV-2 virus to this receptor, the SARS-COV-2 enters the host cell and begins replication therein. Thus, the peptide capable of specifically binding to the ACE2 receptor may play an important role in blocking the invasion of the virus and may contribute to the prevention and treatment of the virus infections. It will be apparent from the experimental results to be described below that the peptide having the sequence as described above may specifically bind to the ACE2 receptor.

In one example, a nucleic acid according to an embodiment of the present disclosure may encode the peptide. In the context of the present disclosure, the dictionary meaning of a nucleic acid is a biochemical substance that stores and conveys genetic information, and includes DNA or RNA. These nucleic acids are components of genes and contain information necessary for the biosynthesis of proteins and peptides. In the context of the present disclosure, the dictionary meaning of the nucleic acid encoding the peptide means that the nucleic acid (DNA or RNA) has genetic information that instructs the synthesis of a peptide composed of a specific amino acid sequence. This information is converted into the peptide via transcription and translation processes. During the transcription process, the genetic information of DNA is copied into mRNA, and during the translation process, the code of mRNA is translated into an amino acid sequence by the ribosome, resulting in the synthesis of the peptide. Through this process, the nucleic acid directly plays a role in determining the structure and function of the peptide.

As known in the art, a combination of nucleic acids encoding the amino acid included in the peptide may vary. Accordingly, according to the present disclosure, there are proposed not only the above-described peptide but also a nucleic acid encoding the above-described peptide, wherein the nucleic acid includes all of theoretical 884,736 nucleic acid sequences encoding the SEQ ID NO: 1, theoretical 393,216 nucleic acid sequences encoding the SEQ ID NO: 2, theoretical 3,981,312 nucleic acid sequences encoding the SEQ ID NO: 3, and theoretical 221,184 nucleic acid sequences encoding the SEQ ID NO: 4. These diverse nucleic acid sequences are attributed to the codon variability of the amino acids. A codon is three consecutive bases of the nucleic acid encoding one amino acid, and multiple codons may encode the same amino acid, so that various nucleic acid sequences for encoding the same peptide sequence may be present. This increases the flexibility of the subject matter of the present disclosure and may help to select nucleic acid sequences optimized for specific biological systems or applications. For example, the nucleic acid sequences may be optimized by taking into account use of the codon that is translated more efficiently in certain species of organisms. This may contribute to enhancing the expression level and stability of the peptide.

In one example, a recombinant expression vector according to an embodiment of the present disclosure may include the nucleic acid. In the context of the present disclosure, the dictionary meaning of the recombinant expression vector is a molecule used to introduce a gene into another cell to express a protein or a peptide. This vector may take the form of plasmids, viruses, artificial chromosomes, etc., and includes essential elements for replication and gene expression, such as selection markers, promoters, reporter genes, etc.

Since the recombinant expression vector includes the nucleic acid, the efficiency and specificity of gene expression may be greatly improved. The nucleic acid incorporated within this vector directs the production of a target protein or peptide within a specific cell type, and thus may be used in medical treatment as well as in scientific research. For example, it plays an important role in a variety of applications, including disease modeling, gene therapy, vaccine development, and mass production of biological agents. In particular, according to the present disclosure, since the specific peptide exhibits high binding specificity to the target protein, the vector corresponding thereto may be an effective tool for preventing or treating SARS-COV-2 virus infection by optimizing the expression of the peptide.

In one example, a cell according to an embodiment of the present disclosure may be transformed with the recombinant expression vector. In the context of the present disclosure, the dictionary meaning of the transformation of the cell is a process of changing the genetic composition of the cell by introducing the DNA from the outside thereto. Through this process, the cell acquires a new gene, which may be expressed in the cell to produce a specific protein.

As the cell is transformed with the recombinant expression vector, continuous production of a specific peptide or protein may be possible. This process allows the cell to receive genetic information and perform a new biological function based on the genetic information. Thus, a purpose of the present disclosure is to produce a peptide that binds to the ACE2 receptor of the SARS-COV-2 virus. The transformed cell may play an important role in a variety of applications, including laboratory studies alone, as well as the production of biologics and the development of gene therapeutics for disease treatment.

In an embodiment, the cell may include one or more cells selected from the group consisting of animal cells, plant cells, yeast, E. coli, and insect cells. In one embodiment, the cells may include one or more cells selected from the group including COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

Using the various cells as described above, the subject matter of the present disclosure has the flexibility to be applied to a wide range of biological systems. Each cell type has its own physiological characteristics and genetic expression profile, which may help optimize the production, stability, and functionality of peptides. The utilization of these different cell types provides an opportunity to develop optimized expression systems for specific uses.

In one example, an antibody or antigen-binding fragment thereof according to an embodiment of the present disclosure is an antibody or antigen-binding fragment thereof capable of specifically binding to the ACE2 receptor, and may include one or more peptide sequences selected from the group consisting of the peptide sequence of SEQ ID NO: 1, the peptide sequence of SEQ ID NO: 2, the peptide sequence of SEQ ID NO: 3, and the peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY |

In the context of the present disclosure, the dictionary meaning of an antibody or antigen-binding fragment thereof is a portion of a protein that may bind to, recognize, and neutralize a specific antigen. The antigen-binding fragment of the antibody is generally responsible for binding with the antigen at a specific site within the variable region of the antibody, and the efficacy and specificity thereof are influenced by the amino acid sequence of the binding region.

In one embodiment, the antibody may be a Fv antibody. In the context of the present disclosure, the dictionary meaning of the Fv antibody is a protein comprising two fragments constituting the variable region of the antibody, namely, the variable region (VL) of the light chain and the variable region (VH) of the heavy chain. The Fv antibody has high specificity and binding power, and may be advantageously used as a therapeutic agent or as a diagnostic tool because of its small size.

In an embodiment, the peptide sequences according to SEQ ID NOs: 1 to 4 may be included in the CDR3 (Complementarity-Determining Region 3) region of the Fv antibody. The CDR3 region of the Fv antibody is the most variable region of the antibody and plays a decisive role in binding to the antigen. This region is an important factor that allows the antibody to have high affinity and specificity to the specific antigen, and is an important determinant that enables effective binding and recognition of the antibody. Thus, the selection and optimization of the peptide sequences contained in the CDR3 region play an important role in maximizing the function of the antibody.

In one example, a composition for preventing SARS-COV-2 according to an embodiment of the present disclosure may include the antibody or antigen-binding fragment thereof. Since the composition for preventing SARS-COV-2 comprises the antibody or antigen-binding fragment thereof, the principle of preventing SARS-COV-2 is that the antibody or antigen-binding fragment thereof prevents the interaction between the spike protein of the virus and the ACE2 receptor of the host cell. The spike protein plays an essential role for the virus to bind to and invade the host cell, and once this binding is blocked, the virus cannot enter the cell and begin to replicate. Thus, the antibody or antigen-binding fragment thereof in accordance with the present disclosure specifically binds to a main binding site of the virus, thereby interfering with the effective binding of the virus's receptor binding domain (RBD) to the ACE2 receptor on the host cell, thereby stopping the initial invasion of the virus and subsequent infection cycles.

The composition for preventing SARS-COV-2 according to an embodiment of the present disclosure includes the antibody or antigen-binding fragment thereof. However, the present disclosure does not exclude the addition of other compositions that may be included in the composition for prevention to the composition for preventing SARS-COV-2 according to an embodiment of the present disclosure. For example, the composition for prevention may include an adjuvant for enhancing an immune response, a stabilizer and a preservative for maintaining the stability of antibodies and peptides, a solubilizer and a buffer for controlling the pH of the composition and improving solubility thereof, and various immune response enhancing agents for enhancing an immune response. As the adjuvants, substances such as aluminum salts and squalene may be used to increase the immunogenicity against the antigen and provide long-term immune protection. Sugar such as sucrose or trehalose may be used as the stabilizer, and thimerosal (sodium ethylmercurithiosalicylate) or paraben may be used as the preservative. In addition, buffer solutions such as phosphate buffer solution (PBS) or tris buffer are used to adjust the pH of the composition and improve solubility thereof. The immune response enhancing agent may contribute to enhancing immune memory and increasing protective effects.

Hereinafter, examples of the present disclosure will be described. However, the examples as described below are only some implementations of the present disclosure, and the scope of the present disclosure is not limited to the following examples.

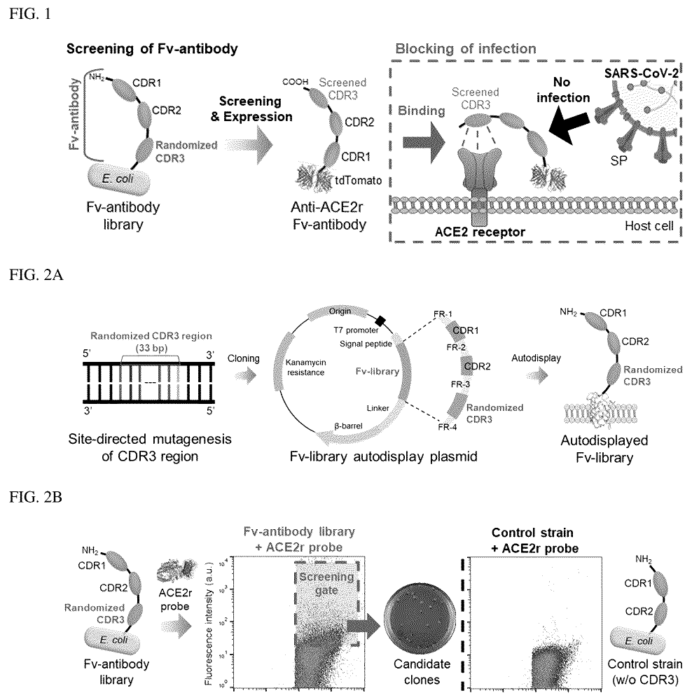

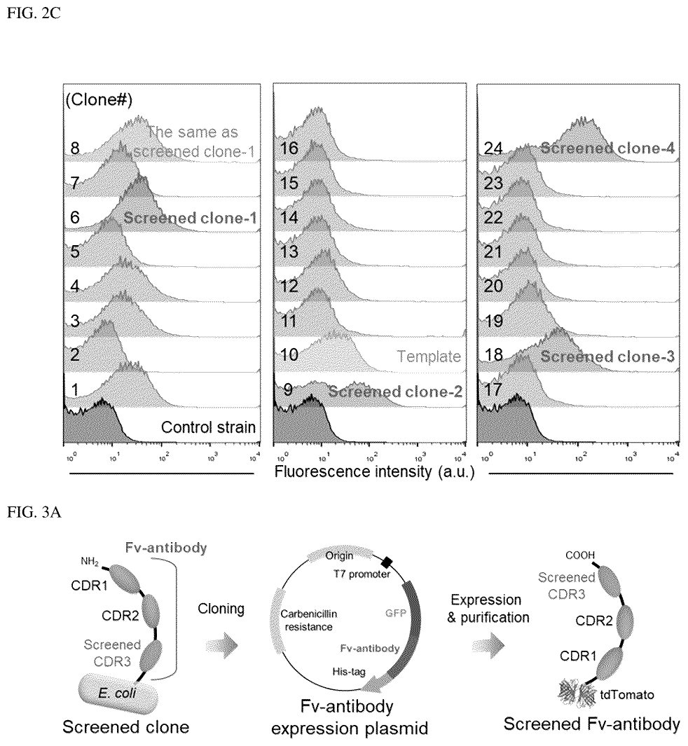

FIG. 2A Screening Fv-antibodies from the Fv-antibody library with the binding affinity to the ACE2 receptor: Preparation of the Fv-antibody library through site-directed mutagenesis and autodisplay of the library on the outer membrane of E. coli. FIG. 2B Screening Fv-antibodies from the Fv-antibody library with the binding affinity to the ACE2 receptor: Screening of target clones with binding affinity to the ACE2 probe using a screening gate. FIG. 2C Screening Fv-antibodies from the Fv-antibody library with the binding affinity to the ACE2 receptor: Flow cytometric analysis of the ACE2 probe activity in screened clones.

The Fv-antibody library was expressed on the outer membrane of Escherichia coli (E. coli) using autodisplay technology as shown in FIG. 1A. Fv-antibodies against the ACE2 receptor were screened from the Fv-antibody library using the binding domain of the ACE2 receptor as a screening probe. The binding domain of the ACE2 receptor (amino acids 1-130) was produced as a fusion protein (43 kDa) with GFP. As shown in FIG. 2B, the Fv-antibody library was reacted by mixing with the ACE2 receptor probe, and E. coli cells showing a high-fluorescence signal were isolated using flow cytometry and cultured on an LB agar plate. When the Fv-antibody library and the ACE2 receptor probe were reacted, E. coli cells with high fluorescence were observed on the flow cytogram. The control strain with autodisplayed CDR1 and CDR2 (without CDR3) exhibited few E. coli cells in the same high-fluorescence region as the Fv-antibody library with three CDRs. These results indicate that the Fv-antibody library contained target E. coli cells with Fv-antibodies with high affinity for ACE2 receptors; this binding affinity results from the CDR3 region of the target E. coli cells. To isolate the target E. coli cells using flow cytometry, the high-fluorescence region of the flow cytogram was set as the gate for isolation, and E. coli cells with a strong fluorescence signal were isolated from the Fv-antibody library. Among the isolated E. coli clones grown on agar plates, the binding affinity of randomly selected clones to the screening probe was estimated, as shown in FIG. 2C. After oligonucleotide sequencing of CDR3, four target clones (6, 9, 18, and 24) were selected as targets. Clone 8 had the same sequence as clone 6, and clone 10 had the same sequence as the template (CDR3 sequence prior to site-directed mutagenesis). Information on these clones (oligonucleotide and amino acid sequences as well as binding affinity) is summarized in Table 1.

TABLE 1

| | | | Binding | |
|---|---|---|---|---|
| | | CDR3 | constant | |
| Screened | Oligonucleotide | sequence | K_{D} (nM) | |
| clone | sequence (33 bp) | (11 mer) | FACS | SPR |
| 1 | 5-GAT ATA AGC GGA GAG | ¹GHPVN | 627 | 26 |
| | AAA CAC CCC GCG GAT | ⁵SVLLD | | |
| | TTC-3 | ¹¹F | | |
| 2 | 5-GAC GGT ACA CCG AAA | ¹GHPRV | 532 | 33 |
| | GGC CCT AGA AAT GAT | FNVGGD | | |
| | TTC-3 | ¹¹F | | |
| 3 | 5-GAC CCT CCA ACT GGT | ¹GVLGP | 665 | 33 |
| | ACA ATG GTA ACA GAT | ⁵RLLID | | |
| | TTT-3 | ¹¹Y | | |
| 4 | 5-TAC GGA AGT CTA CAA | ¹DGPIN | 674 | 42 |
| | GCA CAA GCA CCT GAT | ⁵RTTID | | |
| | GAT-3 | ¹¹Y | | |

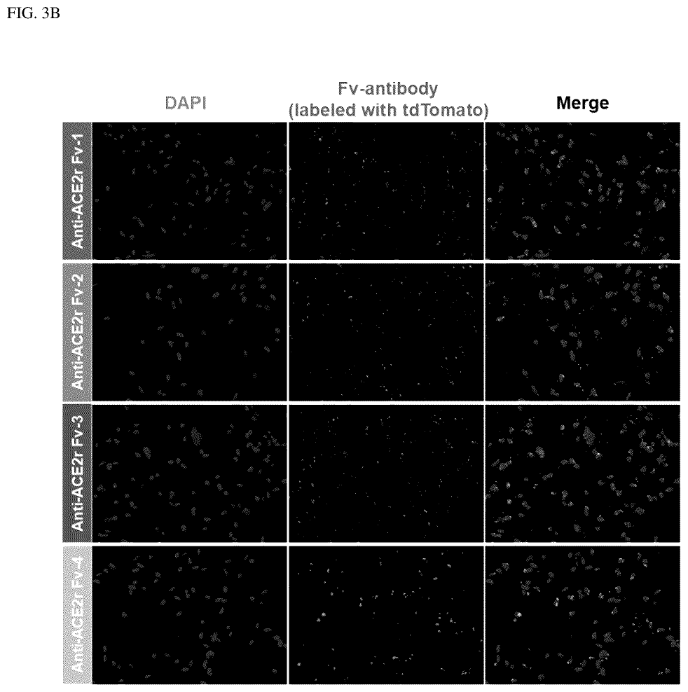

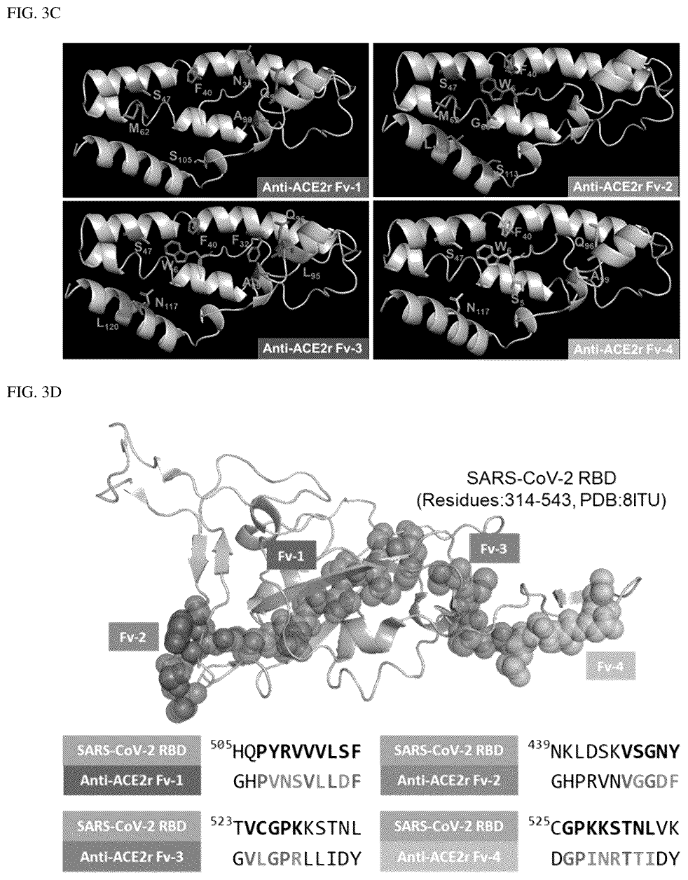

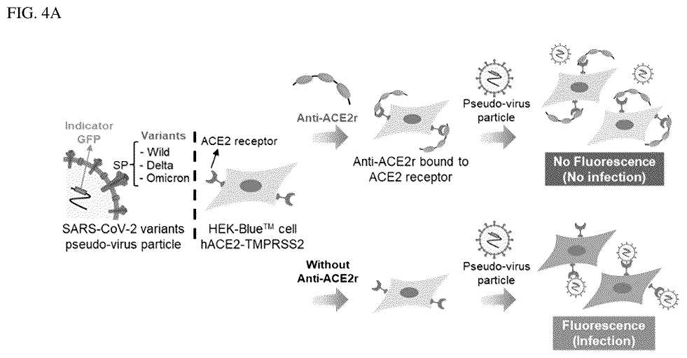

The Fv-antibodies of the four screened clones were expressed as soluble fusion proteins of tdTomato with a molecular weight of 68.2 kDa, as shown in FIG. 3A. The binding affinities (K_{D}) of the four Fv-antibodies to the ACE2 receptor were estimated by using a SPR biosensor. The K_{D} was calculated as 26 nM for anti-ACE2r Fv-1 (clone 6), 33 nM for Fv-2 (clone 9), 33 nM for Fv-3 (clone 18), and 42 nM for Fv-4 (clone 24). As the binding affinity (K_{D}) to the ACE2 receptor was reported to be 31-100 nM for SARS-COV-1 SP and 4.7-10 nM for SARS-COV-2 SP, these results indicate that the four Fv-antibodies exhibited comparable binding affinities to the ACE2 receptor, aligning with the reported values for the SARS-COV-2 SP. First, the binding of the Fv-antibodies to ACE2 receptors on host cells was estimated using HEK293T cells overexpressing the ACE2 receptor and TMPRSS2. After the four expressed Fv-antibodies (combined with tdTomato) were incubated with HEK293T cells, fluorescence images were taken, and the fluorescence signal was observed on the surface of HEK cells, as shown in FIG. 3B. These results show that the four Fv-antibodies specifically bound to the ACE2 receptors of HEK cells.

FIG. 3A Binding analysis of expressed Fv-antibodies: Expression of screened Fv-antibodies. FIG. 3B Binding analysis of expressed Fv-antibodies: Binding affinity of expressed Fv-antibodies to cells that overexpressed ACE2 receptor. FIG. 3C Binding analysis of expressed Fv-antibodies: Binding sites of Fv-antibodies on ACE2 receptors. FIG. 3D Binding analysis of expressed Fv-antibodies: Homology analysis between SARS-COV-2 RBD and anti-ACE2r Fv-antibodies.

As shown in FIG. 3C, docking simulations using AutoDock Vina software from Scripps Research (La Jolla, CA, USA) were employed to study the interaction between the screened Fv-antibodies and the ACE2 receptor (PDB ID: 7U0N). The interaction between the ACE2 receptor and anti-ACE2r Fv-1 exhibited a Gibbs free energy of-7.3 kcal/mol. Amino acids F40, M62, and A99 in the ACE2 receptor participated in hydrophobic interactions, while N33, S47, Q96, and S105 were involved in hydrogen bonding. The interaction between the ACE2 receptor and Fv-2 took place in different regions compared to the interaction with Fv-1 and exhibited a Gibbs free energy of-7.7 kcal/mol. The ACE2 receptor amino acids involved in hydrophobic interactions with Fv-2 were W6, F40, M62, and L120, whereas S47, G66, and S113 participated in hydrogen bonding. The interaction between the ACE2 receptor and Fv-3 took place in distinct regions compared with the previous Fv-antibodies, with a Gibbs free energy of −8.7 kcal/mol. The ACE2 receptor amino acids engaged in hydrophobic interactions with Fv-3 were F32, F40, L95, A99, and L120, whereas W6, S47, Q96, and N117 were involved in hydrogen bonding. The interaction between the ACE2 receptor and Fv-4 occurred at different sites on the ACE2 receptor with a Gibbs free energy of −8.5 kcal/mol. Amino acids involved in hydrophobic interactions were F40 and A99, while S5, W6, S47, Q96, and N117 participated in hydrogen bonding. Docking analysis demonstrated that the four Fv-antibodies (anti-ACE2r Fv-1, -2, -3, and -4) specifically interacted with distinct regions of the ACE2 receptor, and the predicted binding affinity according to docking simulations was comparable to the Gibbs free energy of the four Fv-antibodies. As SP specifically binds to ACE2 receptors on the host cell surface, the amino acid sequences of the Fv-antibodies were compared to those of the RBD for SARS-COV-2 infection. Homology analysis between the amino acid sequences of selected Fv-antibodies and the SARS-COV-2 RBD (PBD ID: 6XC2) was performed using the SIM Alignment Tool (https://web.expasy.org/sim/). The results indicated that four types of Fv-antibodies had amino acid sequences that were either identical or similar to those of the SARS-COV-2 RBD, but in different regions (FIG. 3D)). Compared with the amino acid sequence of the SARS-COV-2 RBD, the Fv-antibodies had identical amino acids (highlighted in red) within the CDR3 region, consisting of 11 residues, as well as amino acids (highlighted in blue) with similar chemical properties. These results strongly suggest that the specific affinity of Fv-antibodies for the ACE2 receptor results from similarity in the amino acid sequence to the SARS-COV-2 RBD. The binding affinities obtained under different measurement conditions were directly compared, and Fv-antibodies were estimated to have similar binding affinity levels to both the SARS-COV-2 RBD and the ACE2 receptor. According to amino acid sequence analysis, the high binding affinity of the screened Fv-antibodies was attributed to homology in the amino acid sequence with these receptors.

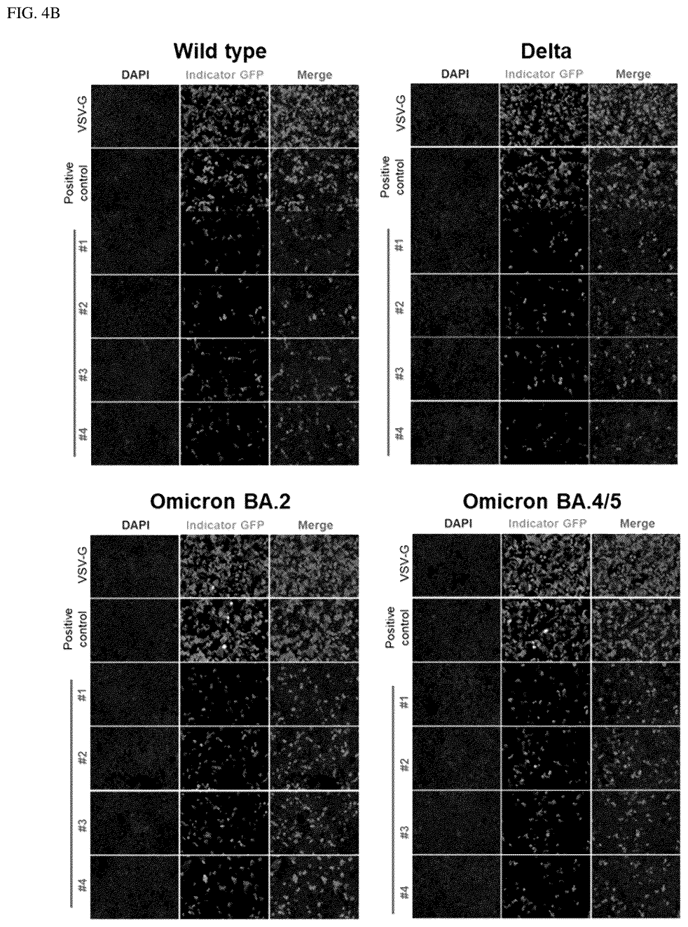

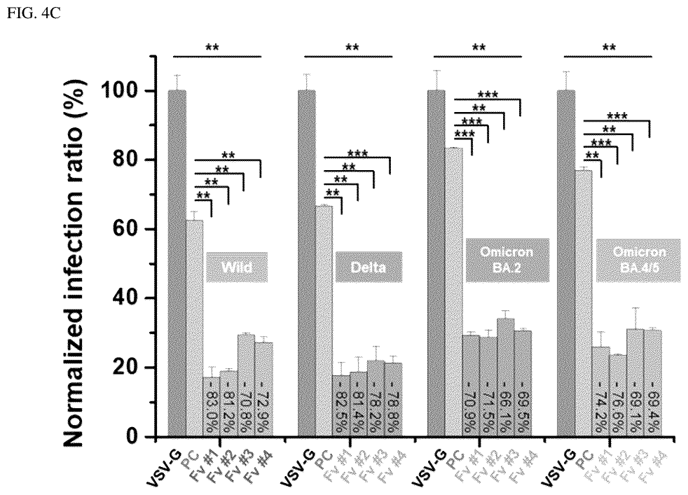

FIG. 4A In vitro cell-based infection assay: (a) In vitro cell-based infection assay using cells that overexpress the ACE2 receptor and pseudo-virus particles with SP from SARS-COV-2 variants. FIG. 4B In vitro cell-based infection assay: Neutralizing activity of screened Fv-antibodies. The concentration of Fv-antibodies was set to their binding affinity (KD), and the Fv antibodies were incubated for 48 h. FIG. 4C In vitro cell-based infection assay: Estimation of the inhibition ratio of SARS-COV-2 infection. Statistical significance is denoted as follows: *P<0.05, **P<0.01, and ***P<0.001.

To estimate the neutralizing activity of Fv-antibodies against SARS-COV-2 infection, an in vitro cell-based infection assay was performed using pseudo-virus particles expressing the SP of SARS-COV-2 and HEK cells overexpressing the ACE2 receptor and TMPRSS2 as the host cell line. In vitro cell-based assays using pseudo-virus particles have been demonstrated to have a strong correlation to the assays with live SARS-COV-2 virus.39-41 Pseudo-virus particles were prepared using a lentivirus, and the SP of SARS-COV-2 was expressed on the virus surface. Additionally, an indicator gene was integrated into the pseudo-virus particles, which could be expressed after the infection of host cells. Therefore, the cell-based infection assay generated a fluorescence signal upon infection of host cells with the pseudo-virus particles, as shown in FIG. 4A. When infection with pseudo-virus particles was blocked by the binding of Fv-antibodies, a fluorescence signal was not generated, and the neutralizing activity of the Fv-antibodies was estimated by comparing the fluorescence signals. The cell-based infection assay was performed for pseudo-virus particles with the SP of SARS-CoV-2 variants, such as Wild-type D614, Delta B.1.617.2, Omicron BA.2, and Omicron BA.4/5. The in vitro cell-based infection assay was conducted to compare the neutralizing activity of these Fv-antibodies at concentrations of Fv-antibodies corresponding to their binding affinity (K_{D}). As shown in FIG. 4B, the four expressed Fv-antibodies (combined with tdTomato) were reacted with HEK-blue cells overexpressing the ACE2 receptor and TMPRSS2, and the fluorescence signal was observed by using a fluorescence microscope. Fluorescence signals were observed for different pseudo-virus particles with the SP of the four SARS-COV-2 variants, which represented binding of the four Fv-antibodies to the ACE2 receptors of HEK-blue cells. Compared with the positive control, which represented infection of pseudo-virus particles without Fv-antibody treatment, the four SARS-COV-2 variants showed far lower fluorescence signals after Fv-antibody treatment. These results indicated that four Fv-antibodies exhibited neutralizing activity against pseudo-viruses particles expressing the SP of SARSCOV-2 variants. Analysis of the fluorescence signals from host cells using flow cytometry was used to quantitatively estimate the infection ratio after treatment with Fv-antibodies (combined with tdTomato), compared with that of vesicular stomatitis virus-G (VSV-G). As shown in FIG. 4C, the infection ratio of the pseudo-virus particle with the SP of SARS-COV-2 (Wild) was reduced by as much as −83% for anti-ACE2r Fv-1, −81% for Fv-2, −70% for Fv-3, and −72% for Fv-4. These results indicate that Fv-antibodies can prevent the infection of SARS-COV-2 by blocking the ACE2 receptor on host cells. For other pseudo-virus particles with the SP from SARS-COV-2 variants, the infection ratio was decreased in a similar range of −70 to −80%, suggesting that infection can be prevented even when mutations are present in the SP.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

TABLE 2

| Sequence | | | | |
|---|---|---|---|---|
| ID | Sequence | Molecule | | |
| Number | Name | Type | Organism | Sequence |
| 1 | peptide_1 | AA | Synthetic | GHPVNSVLLDF |
| | | | construct | |
| 2 | peptide_2 | AA | Synthetic | GHPRVNVGGDF |
| | | | construct | |
| 3 | peptide_3 | AA | Synthetic | GVLGPRLLIDY |
| | | | construct | |
| 4 | peptide_4 | AA | Synthetic | DGPINRTTIDY |
| | | | construct | |
| 5 | CDR1 | AA | synthetic | TYGIQ |
| | | | construct | |
| 6 | CDR2 | AA | synthetic | WIHAGTGGTKY |
| | | | construct | SRKFQG |

What is claimed is:

1. A peptide capable of specifically binding to an ACE2 (angiotensin-converting enzyme 2) receptor, the peptide comprising at least one peptide sequence selected from a group consisting of a peptide sequence of SEQ ID NO: 1, a peptide sequence of SEQ ID NO: 2, a peptide sequence of SEQ ID NO: 3, and a peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY. |

2. A nucleic acid coding the peptide according to claim 1.

3. A recombinant expression vector including the nucleic acid according to claim 2.

4. A cell transformed with the recombinant expression vector according to claim 3.

5. The cell of claim 4, wherein the cell includes at least one cell selected from the group consisting of animal cells, plant cells, yeast, E. coli, and insect cells.

6. The cell of claim 5, wherein the cell includes at least one cell selected from the group consisting of COS7 (monkey kidney cells) cells, NSO cells, SP2/0 cells, CHO (Chinese hamster ovary) cells, W138, BHK (baby hamster kidney) cells, MDCK, myeloma cell lines, HuT 78 cells and HEK293 cells, Escherichia coli, Bacillus subtilis, Streptomyces sp., Pseudomonas sp., Proteus mirabilis or Staphylococcus sp., Aspergillus sp., Pichiapastoris, Saccharomyces cerevisiae, Schizosaccharomyces sp., and Neurosporacrasa.

7. An antibody or antigen-binding fragment thereof capable of specifically binding to an ACE2 receptor, wherein the antibody includes a Fv antibody, wherein a CDR3 region of the Fv antibody includes at least one peptide sequence selected from the group consisting of a peptide sequence of SEQ ID NO: 1, a peptide sequence of SEQ ID NO: 2, a peptide sequence of SEQ ID NO: 3, and a peptide sequence of SEQ ID NO: 4:

| [SEQ ID NO: 1] | |
|---|---|
| | GHPVNSVLLDF |
| [SEQ ID NO: 2] | |
| | GHPRVNVGGDF |
| [SEQ ID NO: 3] | |
| | GVLGPRLLIDY |
| [SEQ ID NO: 4] | |
| | DGPINRTTIDY. |

8. A composition for preventing SARS-COV-2, the composition comprising the antibody or antigen-binding fragment thereof according to claim 7.


SEQUENCE LISTING (6 sequences)

SEQ ID NO: 1 - protein, 11 aa, Synthetic construct
GHPVNSVLLDF

SEQ ID NO: 2 - protein, 11 aa, Synthetic construct
GHPRVNVGGDF

SEQ ID NO: 3 - protein, 11 aa, Synthetic construct
GVLGPRLLIDY

SEQ ID NO: 4 - protein, 11 aa, Synthetic construct
DGPINRTTIDY

SEQ ID NO: 5 - protein, 5 aa, synthetic construct
TYGIQ

SEQ ID NO: 6 - protein, 17 aa, synthetic construct
WIHAGTGGTKYSRKFQG